(19) United States
(12) Patent Application Publication
(10) Pub. No.: US 2009/0022700 A1
(43) Pub. Date: Jan. 22, 2009

(54) Cosmetic water-soluble film

(75) Inventors: Guillaume Cassin, Villebon Sur Yvette (FR); Jean-Thierry Simonnet, Cachan (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/081,909

(22) Filed: Apr. 23, 2008

Related U.S. Application Data
(60) Provisional application No. 60/941,735, filed on Jun. 4, 2007.

(30) Foreign Application Priority Data
Apr. 27, 2007 (FR) 07 54766

(51) Int. Cl. A61K 35/74; A61K 35/66; A61Q 19/00

(52) U.S. Cl. 424/93.42; 424/93.1; 424/93.51; 424/93.5; 424/93.4; 424/93.44; 424/93.46; 424/93.45

(57) ABSTRACT

The present invention relates to a composition which is in the form of a water-soluble anhydrous film comprising at least one water-soluble or water-dispersible film-forming polymer, and at least one live, in particular probiotic, microorganism. It also relates to a kit containing such a composition, as a combination with a secondary composition.

This non provisional application claims the benefit of French Application No. 07 54766 filed on Apr. 27, 2007 and U.S. Provisional Application No. 60/941,735 filed on Jun. 4, 2007.

The present invention relates to the field of cosmetic and/or dermatological care of keratin materials, and more particularly of the skin.

More particularly, it aims to provide compositions dedicated to the care of keratin materials and using microorganisms.

The integration, into galenical formulations, of microorganisms such as probiotics, which are either live or in inactivated form, is particularly advantageous for the dermocosmetic field, due to the benefits that these microorganisms are capable of giving the host organism.

Thus, documents US 2006/002910, US-2003/049231, WO-A-97/36603, U.S. Pat. No. 6,905,692 and U.S. Pat. No. 6,723,326 propose to use specific bacterial strains for their antifungal and/or bactericidal properties in compositions for cosmetic or pharmaceutical use. In general, the microorganisms are incorporated into the corresponding compositions in a live or inactivated form.

However, the introduction of microorganisms in a live form into a composition raises difficulties, in particular related to the biological nature of these active agents.

Thus, the introduction of live microorganisms into galenical compositions means that the latter must be devoid of preservatives. However, for obvious reasons, it then becomes very difficult to guarantee the microbiological cleanliness of the corresponding composition, under normal conditions of use and over a reasonable time scale, which can stretch from several days to several weeks.

In addition, one can understand, with regard to the above observations, that these restrictions, related to the biological nature of the microorganisms, conflict with the use of the latter at high concentrations in compositions.

Consequently, the use of live microorganisms in compositions, in particular for cosmetic or dermatological purposes, poses a problem in terms of the formulation.

The present invention aims precisely to propose a solution to this galenical problem.

Unexpectedly, the inventors have observed that it is possible to give satisfaction in these terms by packaging living organisms, in particular of probiotic type, in a cosmetic composition which is in the form of an anhydrous film.

Alongside the usual cosmetic products which are in liquid, gelled or solid form, it is already known practice to use cosmetic preparations in the form of thin water-soluble films, as described, for example, in document JP-A-2002/0 212 027, or in documents US 2004/09211, US-A-2002/0127254, US-A-2003/0 175 328 and US-A-2003/0 175 333, which describe the production of anhydrous polymeric films for direct administration of cosmetic compositions to previously wetted skin. As regards document US-A-2003/0 186 826, it describes a dry cosmetic composition based on polymers and surfactants, to be administered to the skin or the hair with water. Films containing active agents which are released by placing the films in a liquid such as water or a solvent, for instance oil or ethanol, are also known from document FR-A-2 840 221. Moreover, kits comprising one or more thin anhydrous films and a fluid composition into which this or these film(s) is or are introduced are also known from document US-A-2005/0 249 763. At the time of the application, the thin anhydrous film(s) is (are) dissolved extemporaneously with the fluid composition in order to form a new composition to be applied to the skin, the mucous membranes or the appendages.

However, none of these documents envisions the use of such films for formulating live microorganisms, nor the possibility of introducing the latter into a galenical carrier with a view to extemporaneously forming a composition which takes advantages of the properties of these microorganisms.

According to one embodiment, a subject of the present invention is a composition, hereinafter referred to as composition A, which is in the form of a water-soluble anhydrous film comprising (i) at least one water-soluble or water-dispersible film-forming polymer, and (ii) at least one live, in particular probiotic, microorganism.

According to one embodiment, the present invention is also a composition A in the form of a water-soluble anhydrous film comprising (i) at least one water-soluble or water-dispersible film-forming polymer, and (ii) at least one live, in particular probiotic, microorganism, and comprising at least one oxyalkylenated polydimethylsiloxane derivative.

A subject of the invention is also a cosmetic product obtained by incorporating at least one composition A as defined above, into a second composition B comprising at least one physiologically acceptable medium.

A subject of the invention is also a kit for formulating a cosmetic composition comprising:

i) at least a first composition A which is in the form of a water-soluble anhydrous film and as defined above, and

ii) a second composition B comprising a physiologically acceptable medium.

More particularly, the second composition is other than pure water.

According to a first variant, the product is intended for oral administration and is in particular in the form of a food supplement.

According to a second variant, the product is intended for topical application and is in the form of a cosmetic and/or dermatological composition according to the invention.

This cosmetic product can be formed by extemporaneously adding to composition B, at least all or part of one or more films (composition A). The films added may have an identical or different composition.

According to one embodiment, the final cosmetic product may be obtained by extemporaneously mixing all or part of one or more water-soluble anhydrous films forming a composition A, and an appropriate amount of the second composition B. The term “appropriate amount” is intended to mean an amount such that the film(s) dissolve(s) therein rapidly. This amount can range, for example, from 10 to 1000 mg, preferably from 50 to 800 mg, and better still from 100 to 500 mg.

This mixture may be prepared directly by the user at the time of application. For example, the user dissolves in his or her hand or in any other container provided for this purpose, one or more film(s) in accordance with the invention in the presence of an appropriate amount of the associated composition, or even all of said associated composition.

The appropriate dose of the second composition may also be obtained by using single-dose presentation forms, such as sachets, tubes, vials, prefilled syringes, soft capsules, or shells or trays made of thermoformed plastic. An appropriate dose may also be obtained from a multidose presentation form by using a system that distributes a predefined dose. Such a system may be a pump-dispenser bottle, an aerosol, a graduated pipette or syringe, or a dropper. According to this alternative, the dose may be dispensed into the hand of the user, who directly dissolves the film(s) in the hand just before administration.

According to another specific embodiment, the product may be obtained by incorporating the whole of a film forming a composition A in accordance with the invention into the whole of a second composition B packaged, for example, in a container. The product thus formed may be stored for several days at a temperature capable of guaranteeing its microbiological cleanliness, for example at 4° C., until complete consumption of said product.

The present invention also relates to a process for the cosmetic care of keratin material(s), comprising the topical or oral administration of at least one composition A as defined above, the administration being carried out under conditions suitable for the solubilization of said composition, in particular of the water-soluble or water-dispersible polymer(s) forming said composition.

For the purpose of the present invention, the expression “conditions suitable for its solubilization” is intended to denote conditions under which the film according to the invention is combined with a sufficient amount of water or of aqueous medium to result in its solubilization, the water or the aqueous medium then constituting composition B. The solubilization may be carried out more particularly on contact and in a secondary composition, in particular cosmetic composition, containing an aqueous medium.

Thus, the composition may be for example administered in the form of a product as defined above.

In another embodiment, the solubilization may be carried out by bringing composition A according to the invention into contact with a wetted keratin material, or else at the time of administration by contact with saliva, the water present on the keratin material or the saliva then constituting composition B.

The term “cosmetic composition” denotes a composition for topical application, comprising a cosmetically acceptable medium, i.e. a medium which has a pleasant color, smell and feel and which does not generate unacceptable discomfort (stinging, tautness, redness), liable to dissuade the consumer from using this composition.

The term “physiologically acceptable medium” denotes a nontoxic medium that can be applied to at least one keratin material of human beings.

The term “keratin materials” is intended to cover the skin, the mucous membranes such as the lips, the nails and the keratin fibers, such as the eyelashes and the hair. The cosmetic compositions in accordance with the present invention are particularly advantageous for use on the skin and the lips.

The compositions, products, kit and process according to the invention prove to be particularly advantageous insofar as they are compatible with the use of live microorganisms and make it possible to effectively prevent, over a sustained period of time, any risk of microbiological or bacteriological contamination without requiring the use of preservatives or at least of excessive amounts of preservatives.

They thus also prove to be advantageously suitable for the use of large amounts of microorganisms.

Film

According to the present invention, the term “film” is intended to mean a thin solid. The term “thin” is intended to mean a solid having a thickness of at most 1000 μm.

This film may be gripped, i.e. it generally has a suitable size so that it can be readily handled by the user. It may have a square, rectangular or disc shape, or any other shape. A film generally has a thickness of from 10 μm to 1000 μm, for example from 20 to 500 μm, or for example from 50 to 300 μm. It may have a surface area of from 0.25 to 25 cm², or for example from 2 to 10 cm².

Moreover, according to the present invention, the term “anhydrous film” is intended to mean a film containing less than 15% by weight of water, preferably less than 10% by weight, and better still less than 5% by weight, relative to the total weight of the film, and more preferably containing no water.

In addition, for the purpose of the present invention, the term “water-soluble film” is intended to mean a film which dissolves in water. It is a film composed of one or more water-soluble or water-dispersible polymers.

The term “water-soluble or water-dispersible” is intended to mean polymers having a solubility in water, measured at 25° C., at least equal to 0.1 gram/liter (g/l) (a colored or colorless, macroscopically isotropic and transparent solution being obtained). This solubility is preferably greater than or equal to 1 g/l. The polymers for constituting these films may be of synthetic or natural origins, and, where appropriate, may be modified by chemical reactions. They may or may not be film-forming. These polymers should be physiologically acceptable, i.e. compatible with the skin, the mucous membranes, the hair and the scalp.

The water-soluble polymers used in the film can be of synthetic or natural origin, where appropriate modified by chemical reactions. They are advantageously film-forming.

The term “film-forming polymer” is intended to mean a polymer capable of forming, on its own or in the presence of an auxiliary film-forming agent, a continuous film and preferably a film whose cohesion and mechanical properties are such that said film can be isolated from a carrier.

These polymers are cataloged under the title “Film Formers” in the cosmetic dictionary “International Cosmetic Ingredient Dictionary and Handbook” (see, for example, pages 2903 to 2906 of the ninth edition—2002).

The film-forming polymers may be selected, for example, from the group consisting of:

vinyl polymers, such as polyvinyl acetate, polyvinylpyrrolidones, methyl vinyl ether/maleic anhydride copolymers, vinyl acetate/crotonic acid copolymer, vinylpyrrolidone/vinyl acetate copolymers, vinylpyrrolidone/caprolactam copolymers, polyvinyl alcohols;

film-forming cellulosic derivatives, such as hydroxyethylcellulose, hydroxypropylcellulose, hydroxypropylmethylcellulose, methylcellulose, et hylhydroxyethylcellulose, carboxymethylcellulose and quaternized derivatives of cellulose;

starches and derivatives thereof;

optionally modified polymers of natural origin, such as pullulan, pectin, mannan, galactomannans, glucomannans and their derivatives, gum arabic, guar gum, xanthan gum, karaya gum; alginates, carragheenans, ulvans and other algal colloids; hyaluronic acid and its derivatives; shellac, sandarac gum, dammar resins, elemi resins, copal resins; deoxyribonucleic acid; mucopolysaccharides such as hyaluronic acid or chondroitin sulfate;

anionic, cationic, amphoteric or nonionic polymers derived from chitin or from chitosan,

protein polymers, such as wheat proteins or soybean proteins; keratin and its derivatives, for example keratin hydrolyzates and sulfonic keratins; casein; albumin; collagen; glutelin; glucagon; gluten; zein; gelatins and their derivatives;

acrylic phosphorylcholine copolymers, such as the poly-2-(methacryloyloxyethyl) phosphorylcholine sold under the name Lipidure HM by the company NOF Corporation (INCI name: polyphosphorylcholine glycol acrylate);

anion-cation complexes of gum arabic/gelatin or gum arabic/chitosan or collagen/glycosaminoglycan type;

and mixtures of these polymers.

According to a further embodiment of the invention, the film-forming polymer may be selected from the group consisting of vinyl polymers and cellulosic derivatives, and mixtures thereof.

According to one embodiment the vinyl polymer may be polyvinyl acetate (PVA), which is for example prepared by radical polymerization of the vinyl acetate monomer and then hydrolysis. Use may in particular be made of polyvinyl acetate hydrolyzed at 88%, such as that sold under the name CELVOL 540 PV ALCOHOL by the company Celanese Chemicals.

According to one embodiment, the cellulosic derivatives may be selected from the group consisting of hydroxypropylcellulose (HPC) and hydroxypropylmethylcellulose (HPMC). These polymers are soluble in water and also in organic solvents. This makes it possible to increase the field of solubility of the films containing them. The choice of the molecular weight of these cellulosic polymers should be made judiciously in order to increase the dissolution of the films.

The HPCs that may, for example, be used are those sold by the company Hercules under the name:

Klucel® MF, the molecular weight of which is 850 000 (viscosity 4000-6500 mPa at 2% in water);

Klucel® EF, the molecular weight of which is 80 000 (viscosity 300-600 mPa at 10% in water).

The HPMC that may be, for example, used is the hydroxypropylmethylcellulose having a viscosity of 40-60 cps (40-60 mpa·s) at 2% in water at 20° C., sold by the company Sigma-Aldrich.

According to one embodiment, the film-forming polymer may be selected from the group consisting of polyvinyl acetate, hydroxypropylcellulose and hydroxypropylmethylcellulose, and mixtures thereof.

The amount of water-soluble film-forming polymer(s) in the composition according to the invention may range, for example, from 10% to 95% by weight, for example from 20% to 70% by weight, or for example from 30% to 60% by weight, relative to the total weight of composition A.

It is also possible to use, in the composition of the invention, a polymer which is both a film-forming polymer and a thickening polymer, selected, for example, from the group consisting of cellulosic derivatives and polymers of natural origin which may be both film-forming and thickening. The amount for use remains that indicated above: for example, from 10% to 95% by weight, for example from 20% to 70% by weight, or for example from 30% to 60% by weight, relative to the total weight of said film.

According to a specific embodiment, the composition according to the invention may contains, in addition to the film-forming polymer, at least one polysaccharide thickener.

The polysaccharide thickeners that may be used in the film according to the invention may be selected from the group consisting of polysaccharides with a gelling capacity.

The term “gelling capacity” defines the fact that, at a concentration of greater than or equal to 0.5% by weight in water, the viscosity of the solutions thus obtained is greater than or equal to 0.01 Pa·s for a shear rate equal to 1 s⁻¹, the measurements being carried out at 25° C. using a Haake RheoStress RS150 rheometer in cone/plate configuration, the measurements of the measuring cone being the following: diameter: 60 mm and angle: 2°.

The polysaccharide thickeners may be for selected from the group consisting of gum arabic, gum ghatti, karaya gum, locust bean gum, guar gum, tamarind gum, xanthan gum, gellan, pectins, gum tragacanth, agar, alginates, carragheenan, furcelleran, konjac gum and cellulose derivatives, and mixtures thereof.

According to one embodiment of the invention, the polysaccharide thickeners may be selected from the group consisting of carragheenans, which are linear polysaccharides extracted from certain red algae of the family Rhodophyceae. They are composed of alternating β-1,3 and α-1,4 galactose residues, it being possible for numerous galactose residues to be sulfated. Three types of carragheenans exist, referred to as kappa-carragheenan, iota-carragheenan and lambda-carragheenan. This family of polysaccharides is described, for example, in chapter 3 of the book “Food Gels” edited by Peter Harris, Elsevier 1989, the content of which being herein incorporated by reference.

Use may in particular be made of the carragheenan sold under the name Satiagum UTC 10 by the company Degussa.

The amount of thickener(s) in composition A according to the invention may, for example, range from 0.5% to 40% by weight, for example from 1% to 20% by weight, or for example from 5% to 10% by weight, relative to its total weight.

In addition, according to one advantageous embodiment, composition A which is in the form of a film may incorporate, in combination with the polymer as defined above, at least one oxyalkylenated polydimethylsiloxane derivative. As described in document US 2008/081055, incorporated herein by reference, such a derivative has the advantage of significantly increasing the dissolution kinetics of the water-soluble film which incorporates it.

The oxyalkylenated polydimethylsiloxanes (PDMSs) that may be used according to the invention may be water-soluble or water-dispersible. The term “water-soluble or water-dispersible” is intended to mean PDMSs having a solubility in water, measured at 25° C., at least equal to 0.1 gram/liter (g/l) (a colored or colorless, macroscopically isotropic and transparent solution being obtained). This solubility is preferably greater than or equal to 1 g/l.

These PDMSs may be preferably selected from the group consisting of water-soluble silicones which comprise at least one monovalent, polyoxyalkylenated end or pendent group, and which, when introduced at 0.05% by weight into an aqueous solution, are capable of reducing the surface tension of water to a value of less than 35 mN/m, and preferably less than 30 mN/m.

The oxyalkylenated PDMSs that may be suitable for the invention may be for example selected from the group consisting of water-soluble silicones of general formula (a) below:

R²₃SiO(R²₂SiO)ₚ(R²PESiO)_{q}SIR²₃ (a)

in which:

the radicals R², which may be identical or different, denote a monovalent hydrocarbon-based radical selected from the group consisting of alkyl, aryl and aralkyl radicals containing at most 10 carbon atoms; some of the radicals R² possibly also containing, in addition, an ethylcyclohexylene monoxide group of formula:

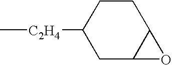

and being in low proportion in the polysiloxane chain;

p ranges from 0 to 150, for example from 0 to 100, or for example from 0 to 30;

q ranges from 1 to 12, for example from 1 to 10, or for example from 1 to 8;

the polyether group PE has the following formula (b):

—CₓH₂ₓ(OC₂H₄)_{y}(OC₃H₆)_{z}OR³ (b)

in which:

x ranges from 1 to 8, for example ranges from 2 to 4, or for example is equal to 3;

y is greater than 0;

z is greater than or equal to 0; the values of y and z being such that the total molecular weight of the polyoxyalkylenated portion of the polyether group PE ranges from 200 to 10 000, or for example from 350 to 4000;

R³ denotes hydrogen, a C₁-C₈ alkyl group or a C₂-C₈ acyl group.

It should be noted that, when z is other than 0, the polyoxyethylene and polyoxypropylene units may be distributed randomly along the polyether chain PE or distributed in blocks or else distributed both in blocks and randomly.

According to one embodiment, the radicals R² may be selected from the group consisting of C₁-C₄ alkyl groups and hexyl, phenyl and benzyl groups. In addition, for example, the radicals R² may be selected from alkyl groups and methyl, ethyl and butyl groups, for example, they denote a methyl radical.

According to one embodiment, the radicals R³ may be selected from the group consisting of C₁-C₄ alkyl groups, and for example denote a methyl radical.

The water-soluble silicones of formula (a) may be obtained according to the process described in document U.S. Pat. No. 4,847,398 incorporated herein by reference.

Among the water-soluble silicones of formula (a), use is for example made of those of formula (a′) below:

MeSiO(MeSiO)ₚ(MePESiO)_{q}SIMe₃ (a′)

in which p and q have the same values as indicated above for formula (a), and Me denotes a methyl radical; PE denotes:

—(CH₂)₃O(OC₂H₄)_{y}(OC₃H₆)_{z}OR³ (b′)

where y and z have the same values as indicated above for formula (b), and R³ denotes hydrogen or a C₁-C₄ alkyl group, and for example a methyl radical.

As other family of water-soluble PDMSs that may be used according to the invention, mention may be made of the branched silicones of formula (c) below:

(MeSiO)_{q-2}[(SiOMe₂)_{p/q}OPE]_{q} (c)

where p and q have the same values as indicated above for formula (a); Me signifies methyl; PE denotes the group of formula (d) below:

—(OC₂H₄)_{y}(OC₃H₆)_{z}R³ (d)

where y and z have the same values as indicated above in formula (b), and R³ denotes a C₁-C₄ alkyl group, and more particularly a methyl radical.

Use may, of course, be made of a mixture of the silicones of formula (a) and of formula (c).

Such oxyalkylenated PDMSs are, for example, sold by the company OSI under the trade names Silwet L-720®, Silwet L-7002®, Silwet L-7600®, Silwet L-7604®, Silwet L-7605®, Silwet L-7607®, Silwet 1614, Silwet L-7657®, Silwet L-7200®, Silwet L7230®, Silsoft 305®, Silsoft 820®, Silsoft 880®, Tego wet 260®, Tegowet 500®, Tegowet 505 and Tegowet 510®.

The following table gives surface tension values at 25° C. for aqueous solutions comprising 0.05% (by weight) of various oxyalkylenated PDMSs:

| | Surface tension at 0.05% |
|---|---|
| Oxyalkylenated PDMS | in water (mN/m) |
| Tegowet 500 ® | 33 |
| Tegowet 510 ® | 29 |
| Silsoft 880 ® | 26 |
| Silsoft 305 ® | 21 |

The amount of oxyalkylenated polydimethylsiloxane(s) in composition A according to the invention may range, for example, from 0.5% to 30% by weight, for example from 1% to 20% by weight, relative to the total weight of the film.

Composition A according to the invention may also comprise one or more plasticizers selected, for example, from the group consisting of polyols such as glycerol, sorbitol, mono- and/or disaccharides, dipropylene glycol, butylene glycol, pentylene glycol or polyethylene glycols such as PEG-400. The amount of plasticizer(s) may range, for example, from 1% to 40% by weight, and better still from 2% to 15% by weight, relative to the total weight of the composition.

Microorganisms and in Particular Probiotic Microorganisms

The microorganisms that may be suitable for the invention are physiologically acceptable. In other words, they are microorganisms which may be administered to animals or humans without any risks.

According to one embodiment, in the present invention, at least one microorganism said to be of probiotic type is used.

For the purpose of the present invention, the term “probiotic microorganism” is intended to mean a live microorganism which, when it is consumed in appropriate amount, has a positive effect on the health of its host “Joint FAO/WHO Expert Consultation on Evaluation of Health and Nutritional Properties of Probiotic in Food Including Powder Milk with Live Lactic Acid Bacteria, 6 Oct. 2001”, and which can in particular improve intestinal microbial balance.

According to a variant of the invention, this microorganism may be used in an isolated form, i.e. not mixed with one or more compound(s) that may be associated with it in its natural environment or its culture medium of origin.

The microorganisms suitable for the invention may be for example selected from the group consisting of ascomycetes such as Saccharomyces, Yarrowia, Kluyveromyces, Torulaspora, Schizosaccharomyces pombe, Debaromyces, Candida, Pichia, Aspergillus and Penicillium, bacteria of the genus Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus or Lactobacillus, and mixtures thereof.

As ascomycetes that may be most particularly suitable for the present invention, mention may for example be made of Yarrowia lipolitica and Kluyveromyces lactis, and also Saccharomyces cereviseae, Torulaspora, Schizosaccharamyces pombe, Candida and Pichia.

As regards the probiotic microorganisms, the following types of bacteria and yeasts may be generally used:

lactic acid bacteria, i.e. which produce lactic acid by fermentation of sugar. They are divided up, according to their morphologies, into two groups:
Lactobacillus species: Lactobacillus acidophilus; amylovorus, casei, rhamnosus, brevis, crispatus, delbrueckii (subsp. bulgaricus, lactis), fermentum, helveticus, gallinarum, gasseri johnsonii, paracasei, plantarum, reuteri, salivarius, alimentarius, curvatus, casei subsp. casei, sake,
Gocci: Enterococcus (faecalis, faecium), Lactococcus lactis (subsp. lactis or cremoris), Leuconstoc mesenteroides subsp. dextranicum, Pediococcus acidilactici, Sporolactobacillus inulinus, Streptococcus salvarius subsp. Thermophilus, Streptococcus thermophilus, Staphylococccus carnosus, Staphylococcus xylosus,

bifidobacteria or Bifidobacterium species, for instance Bifidobacterium adolescentis, animalis, bifidum, breve, lactis, longum, infantis, pseudocatenulatum,

yeasts, for instance Saccharomyces (cerevisiae or else boulardii),

the other sporulated bacteria, for instance Bacillus (cereus var toyo or subtilis), Bacillus coagulans, Bacillus licheniformis, Escherichia coli strain nissle, Propionibacterium freudenreichii,

and mixtures thereof.

Lactic acid bacteria and bifidobacteria are the most commonly used probiotics.

Specific examples of probiotic microorganisms may be Bifidobacterium adolescentis, Bifidobacterium animalis, Bifidobacterium bifidum, Bifidobacterium breve, Bifidobacterium lactis, Bifidobacterium longum, Bifidobacterium infantis, Bifidobacterium pseudocatenulatum, Lactobacillus acidophilus (NCFB 1748); Lactobacillus amylovorus, Lactobacillus casei (Shirota), Lactobacillus rhamnosus (strain GG), Lactobacillus brevis, Lactobacillus crispatus, Lactobacillus delbrueckii (subsp. bulgaricus, lactis), Lactobacillus fermentum, Lactobacillus helveticus, Lactobacillus gallinarum, Lactobacillus gasseri, Lactobacillus johnsonii (CNCM I-1225), Lactobacillus paracasei, Lactobacillus plantarum, Lactobacillus reuteri, Lactobacillus salivarius, Lactobacillus alimentarius, Lactobacillus curvatus, Lactobacillus casei subsp. casei, Lactobacillus sake Lactococcus lactis, Enterococcus (faecalis, faecium), Lactococcus lactis (subsp. lactis or cremoris), Leuconstoc mesenteroides subsp. dextranicum, Pediococcus acidilactici, Sporolactobacillus inulinus, Streptococcus salvarius subsp. Thermophilus, Streptococcus thermophilus, Staphylococccus carnosus, Staphylococcus xylosus, Saccharomyces (cerevisiae or else boulardii), Bacillus (cereus var toyo or subtilis), Bacillus coagulans, Bacillus licheniformis, Escherichia coli strain nissle, Propionibacterium freudenreichii and mixtures thereof.

According to one embodiment, they are probiotic microorganisms derived from the group of lactic acid bacteria, such for example, in particular, Lactobacillus and/or Bifidobacterium.

By way of illustration of these lactic acid bacteria, mention may more particularly be made of Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium lactis, Bifidobacterium infantis, Bifidobacterium adolescentis or Bifidobacterium pseudocatenulatum, and mixtures thereof.

The species most particularly suitable may be Lactobacillus johnsonii, Lactobacillus paracasei, Bifidobacterium adolescentis, Bifidobacterium longum and Bifidobacterum lactis NCC 2818, respectively deposited, according to the Treaty of Budapest, with the Institut Pasteur (28 rue du Docteur Roux, F-75024 Paris cedex 15) on Jun. 30, 1992, Jan. 12, 1999, Apr. 15, 1999, Apr. 15, 1999, Jun. 7, 2005 under the following designations: CNCM I-1225, CNCM I-2116, CNCM I-2168, CNCM I-2170 and CNCM I-3446, and the genus Bifidobacterium longum (BB536), and mixtures thereof.

Microorganisms of thermal plankton type may also be added.

This or these microorganism(s), in particular probiotic microorganism(s), may be combined with at least one live microorganism of skin flora.

By way of illustration of microorganisms of this type, mention may be made of:

Staphylococcus epidermis, S. haemolyticus, S. homonis, S. similans,

Corynobacterium lipophiles, C. jeikeium, C. urealyticum, C. minutissimum,

Propionobacter granulosum, P. avidum,

Micrococcus luteus, M. varians
Streptococcus A, C and G and
Brevibacterium.

In compositions A according to the invention, use will generally be made of 102 to 1015 cfu/g of live microorganism(s) of skin flora, relative to the total weight of the cosmetic composition in film form.

According to another variant of the invention, a microorganism above, and in particular a probiotic microorganism, may be combined with at least one nonphotosynthetic filamentous bacterium or an extract thereof.

By way of illustration of these bacteria, mention may in particular be made of extracts of bacteria prepared from nonphotosynthetic filamentous bacteria as defined according to the classification of Bergey's Manual of Systematic Bacteriology (Vol. 3, sections 22 and 23, 9th edition, 1989 incoporated herein by reference), among which mention may be made of the bacteria belonging to the Beggiatoales order, and more particularly the bacteria belonging to the genera Beggiatoa, Vitreoscilla, Flexithrix or Leucothrix.

The bacteria which have just been defined, and several of which have already been described, generally have an aquatic habitat and can be found in particular in marine waters or in thermal springs. Among the bacteria that can be used, mention may, for example, be made of:

Vitreoscilla filiformis (ATCC 15551)

Vitreoscilla beggiatoïdes (ATCC 43181)

Beggiatoa alba (ATCC 33555)

Flexithrix dorotheae (ATCC 23163)

Leucothrix mucor (ATCC 25107)

Sphaerotilus natans (ATCC 13338).

According to the invention, the term “bacterial extract” is intended to mean an extract of the bacterial biomass or any active fraction of said extract, in particular:

(i) of the bacterial cells isolated from the culture medium, which have been concentrated, for example by centrifugation (“nonstabilized cell extract”); or

(ii) of the bacterial cells which have been concentrated (i), and then subjected to a process to rupture the bacterial cell envelopes, by any means known to those skilled in the art, such as the action of ultrasound or preferably autoclaving (“stabilized cell extract”). The term “envelopes” is intended to mean bacterial wall and possibly underlying membranes;

(iii) the supernatant obtained by filtration of the stabilized cell extract (ii),

or any active fraction of said extract.

These extracts or fractions may be conserved, for example, by freezing said extracts or said fractions, and used after thawing.

The nonphotosynthetic filamentous bacterial extract that may be used in the composition used in the process according to the invention may, for example, be selected from the group consisting of a cell extract, the supernatant of said cell extract or an active fraction of said cell extract.

According to one embodiment, the nonphotosynthetic filamentous bacterial extract may be a cell extract of Vitreoscilla filiformis.

According to one embodiment, an extract of Vitreoscilla filiformis (ATCC 15551) will be used.

In order to prepare the bacterial extract according to the invention, said bacteria may be cultured according to methods known to those skilled in the art, or reference can be made in particular to the description of patent application WO-A-94-02158. A cell extract from which the supernatant may be separated, for example by filtration and centrifugation, may be obtained. The extract may be used in aqueous form or in lyophilized form.

As specified above, the microorganism(s) is (are) used according to the invention in a live form.

Thus, for the purpose of the present invention, a live microorganism form is intended to cover a form which has the ability to multiply provided that it is placed in an environment suitable for the manifestation of this ability. Thus, for the purpose of the present invention, the term “live” covers the “dormancy” state in which the microorganisms can be placed following a physicochemical treatment such as, for example, their formulation in a soluble anhydrous film.

The amount of live microorganism(s) in the composition in the form of a soluble anhydrous film may be between 10⁻¹ cfu/g and 10¹⁵ cfu/g.

According to one embodiment, the microorganisms, in particular probiotic microorganisms, and/or fractions and/or metabolites thereof, may be formulated in an amount equivalent to at least 10¹ cfu/g, for example, at doses ranging from 101 to 10¹⁵ cfu/g, or for example from 10³ to 10¹² cfu/g of the composition containing them.

When the microorganisms or extracts thereof are in the form of an aqueous suspension in which the amount of active material can be between 0.1% and 15%, the amount of this suspension in the composition, before it is formulated into a soluble anhydrous film, may be between 1% and 90%.

It may be useful to add a rehydration protector, which will allow the film to be rehydrated without the microorganism suffering. Such a compound has the effect of protecting the microorganisms against the future rehydration of the anhydrous film in order in particular to prevent osmotic shocks.

By way of example, mention may be made of inositol, mannitol, glucose, sucrose, trehalose, maltose, xylitol, polyvinylpyrrolidone, polyvinyl alcohol, dextrin, maltodextrin and, in general, all monosaccharides and oligosaccharides (2 to 10 units).

To this effect, mention may also be made of starches and modified starches, and also glycols: glycerol, sorbitol, adonitol, propylene glycols, dipropylene glycols and butylene glycol, and also amino acids and oligopeptides (2 to 25) such as glutamates, or aspartates.

Use may also be made of cysteine, ascorbates and erythorbates and also cyclodextrins. They can be used alone or as a mixture.

Mention may also be made of silica and its derivatives, clays, cellulosic derivatives (HEC, HPC, HMPC, etc.), polymers of natural origin such as alginates, xanthans, locust bean gum, guar gums, pectins, agar-agar, carragheenans; polymers of bacterial origin, such as hyaluronic acid, dextran, gellan and hydrogels such as carbomers, AMPS derivatives, etc.

Use may also be made of solid particles and fillers such as:

certain talcs, such as “Talc K1” from the company Nippon or “Talc Extra Steamic OOS” from the company Luzenac;

certain sericites, such as “Sericite BC282” from the company Whittaker;

hydroxyapatite;

silica microspheres with an open porosity, or preferably hollow silica microspheres, such as the “Silica Beads” from the company Maprecos;

the “Macrolite” ceramic or glass microcapsules from the company 3M;

microporous polymer microspheres, which have a structure similar to that of a sponge, such as those made of crosslinked acrylate copolymer “Polytrap” from the company Dow Corning, and those of poly(methyl methacrylate) “Micropearl M” or “Micropearl M 100” from the company Seppic;

polymer microcapsules which comprise a single closed cavity and form a reservoir, which can contain a liquid, in particular an active cosmetic agent; they are prepared by known processes such as those described in patents U.S. Pat. No. 3,615,972 and U.S. Pat. No. 4,397,799 which are herein incorporated by reference. They may, for example, be made of polymers or copolymers of ethylenically unsaturated acid, amine or ester monomers, of urea-formaldehyde polymers, or of vinylidene chloride polymers or copolymers; by way of example, mention may be made of the microcapsules made of methyl acrylate or methyl methacrylate polymers or copolymers, or else of copolymers of vinylidene chloride and of acrylonitrile; among the latter, mention will in particular be made of those which contain, by weight, 20-60% of units derived from vinylidene chloride, 20-60% by weight of units derived from acrylonitrile and 0-40% by weight of other units, such as units derived from an acrylic and/or styrene monomer; use may also be made of acrylic polymers or copolymers crosslinked, for example in the case of polymers comprising a carboxylic group, with diols acting as crosslinking agents; by way of example, mention may be made of the vinylidene chloride-acrylonitrile copolymer microcapsules “Expancel” from the company Kemanord Plast, the “Q-Max” microcapsules from the company Q-Max and the “3M” microcapsules from the company.

The amount of rehydration protector(s) in the composition to be dehydrated may range from 1% to 80%.

As for the anhydrous film form of composition A, it can comprise from 1% to 70% by weight, for example from 5% to 60% by weight, for example from 10% to 50% by weight of rehydration protector(s) by weight of the total weight of said “anhydrous film” form in the dry state.

Preparation of the Film-Type Composition Comprising at Least One Microorganism

The film forming composition A according to the invention comprising at least one live microorganism should be prepared under conditions such that they do not kill the microorganism(s).

For example, such a film may be prepared in two steps, the first being dedicated to the preparation of the mixture that must be converted into the form of a film, and the second being dedicated, specifically, to the formation of the film from the above mixture.

During the first step, the prior dispersion, with stirring, of the water-soluble or water-dispersible polymers in water is carried out. The whole mixture is kept stirring, preferably vigorously, until a homogeneous gel is obtained. This stirring may, for example, be adjusted to a speed of 1200 to 1600 rpm, with in particular a turbine mixer, for example of Rayneri type.

The microorganism(s) under consideration is (are) subsequently introduced, along with the other compounds If present, such as, for example, an oxyalkylenated polydimethylsiloxane derivative such as the PEG-12 dimethicone sold under the name Silsoft 880, the advantage of which is to improve the solubilization rate of the anhydrous film, and also, for example, glycerol, PVP and glucose as rehydration protectors. The introduction of each of the compounds may be carried out under conditions which ensure that they are dispersed homogeneously in the mixture, while taking care not to destructure the gel and while preserving its homogeneity in terms of composition.

To prepare the corresponding film, this mixture may be coated onto a carrier, using a film drawer. The apparatus used may be a film-applying apparatus from Braive Instruments. The wet thickness of the deposit, i.e. the thickness at application of the composition, may range from 25 μm to 2000 μm and is generally of the order of 500 pm.

The film thus obtained may be then placed in a drying oven, the drying temperature being adjusted so as not to kill the live microorganisms.

According to the variants of the invention, the compositions in film form can be administered topically or orally.

When these compositions are used, they must be placed in the presence of a sufficient amount of water or of an aqueous medium to ensure the solubilization of the water-soluble polymer(s) that they contain.

This water or aqueous medium may be in various galenical forms, according to the method of administration selected.

The galenical carrier may be of various nature, according to the type of composition under consideration.

For example, in the case of oral administration, the composition according to the invention may be introduced into any fluid food carrier provided that it is suitable for the dissolution of the water-soluble or water-dispersible polymer, such as, for example, milk, yoghurt, cheese, fermented milks, drinks, mineral waters or soups. The composition according to the invention may also be intended for animals.

The compositions according to the invention may thus be formulated with the usual excipients and constituents for such oral compositions or food supplements, i.e., in particular, fatty and/or aqueous constituents, humectants, thickeners, preservatives, texturing, flavoring and/or coating agents, antioxidants, preservatives and dyes that are customary in the food sector.

The formulating agents and excipients for oral composition, also referred to as composition B, and in particular for food supplements, are known in this field and are not the subject of a detailed description herein.

Of course, compositions B intended for oral administration according to the invention can contain several other active agents.

By way of active agents that can be used, mention may be made of vitamins B3, B5, B6, B8, C, E, or PP, carotenoids, curcuminoids and niacin.

In particular, use may be made of an antioxidant complex comprising vitamins C and E, and at least one carotenoid, in particular a carotenoid chosen from β-carotene, lycopene, astaxanthine, zeaxanthine and lutein, flavonoids such as catechins, hesperidin, proanthocyanidins and anthocyanins.

The composition of type B may advantageously comprise at least one prebiotic or a mixture of prebiotics. More particularly, these prebiotics may be selected from the group consisting of oligosaccharides, produced from glucose, galactose, xylose, maltose, sucrose, lactose, starch, xylan, hemicellulose, inulin, gums, for example of acacia type, or a mixture thereof. More particularly, the oligosaccharide comprises at least one fructooligosaccharide. More particularly, this prebiotic may comprise a mixture of fructooligosaccharide and inulin.

For their part, the compositions of type A for topical application may be formulated with a secondary composition of type B which may be for example in the form of aqueous, aqueous-alcoholic or oily solutions, of dispersions of the lotion or serum type, of suspensions or emulsions obtained by dispersion of a fatty phase in an aqueous phase (O/W) or vice versa (W/O), or of suspensions or emulsions of liquid or semiliquid consistency, of the milk type, or of soft, semisolid or solid consistency, of the cream type, of aqueous gels or else of microemulsions.

According to a preferred variant of the invention, the associated composition B, referred to as second composition, may be a cosmetic and/or dermatological composition, in other words a composition capable of making up and/or providing care for a keratin material such as, for example, the skin, the lips or the head of hair. Such a composition is then different than pure water.

For example, such a composition may comprise at least one compound selected from the group consisting of thickeners, gelling agents, emulsifiers, dyestuffs and/or organic or inorganic fillers.

The compositions, for example cosmetic and/or dermatological compositions, of type B under consideration according to the invention may advantageously contain at least one liquid fatty phase.

Advantageously, they may be in the form of an emulsion.

The compositions B according to the invention may thus be, for example, in the form of an emulsion obtained by dispersion of an aqueous phase in a fatty phase (W/O) or of a fatty phase in an aqueous phase (O/W), of liquid or semiliquid consistency, of the milk type, or of soft, semisolid or solid consistency, of the cream or gel type, or else of a multiple emulsion (W/O/W or O/W/O). These compositions may be prepared according to the usual methods.

According to one embodiment variation, the second composition B may, for example, be in the form of an oil-in-water emulsion, especially obtained according to the phase-inversion temperature method according to PIT technology.

The emulsions generally may contain at least one emulsifier selected from the group consisting of amphoteric, anionic, cationic or nonionic emulsifiers, used alone or as a mixture. The emulsifiers are chosen appropriately according to the emulsion to be obtained (W/O or O/W). The emulsifiers may be generally present in the composition in a proportion that may range, for example, from 0.3% to 30% by weight, for example from 0.5% to 20% by weight, relative to the total weight of the composition.

For the O/W emulsions, emulsifiers that may be mentioned include, for example, nonionic surfactants, and in particular esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives comprising oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose, alkylglucose) esters of C₈-C₂₄ fatty acids, and the oxyalkylenated derivatives thereof; fatty alcohol ethers; the sugar ethers of C₈-C₂₄ fatty alcohols, and mixtures thereof.

As glyceryl esters of fatty acids, mention may in particular be made of glyceryl stearate (glyceryl mono-, di- and/or tristearate) (INCI name: glyceryl stearate) or glyceryl ricinoleate, and mixtures thereof.

As polyethylene glycol esters of fatty acids, mention may in particular be made of polyethylene glycol stearate (polyethylene glycol mono-, di- and/or tristearate), and more especially polyethylene glycol 50 EO monostearate (INCI name: PEG-50 stearate) and polyethylene glycol 100 EO monostearate (INCI name: PEG-100 stearate), and mixtures thereof.

Mixtures of these surfactants may also be used, for instance the product containing glyceryl stearate and PEG-100 stearate, sold under the name Arlacel 165 by the company Uniqema, and the product containing glyceryl stearate (glyceryl monodistearate) and potassium stearate, sold under the name Tegin by the company Goldschmidt (INCI name: glyceryl stearate SE).

As fatty acid esters of glucose or of alkylglucose, mention may in particular be made of glucose palmitate, alkylglucose sesquistearates, for instance methylglucose sesquistearate, alkylglucose palmitates, for instance methylglucose palmitate or ethylglucose palmitate, fatty esters of methylglucoside, and more especially the diester of methylglucoside and of oleic acid (INCI name: methyl glucose dioleate); the mixed ester of methylglucoside and of the oleic acid/hydroxystearic acid mixture (INCI name: methyl glucose dioleate/hydroxystearate); the ester of methylglucoside and of isostearic acid (INCI name: methyl glucose isostearate); the ester of methylglucoside and of lauric acid (INCI name: methyl glucose laurate); the mixture of the monoester and diester of methylglucoside and of isostearic acid (INCI name: methyl glucose sesquiisostearate); the mixture of the monoester and diester of methylglucoside and of stearic acid (INCI name: methyl glucose sesquistearate), and in particular the product sold under the name Glucate SS by the company Amerchol, and mixtures thereof.

As oxyethylenated ethers of a fatty acid and of glucose or of alkylglucose, mention may, for example, be made of the oxyethylenated ethers of a fatty acid and of methylglucose, and in particular the polyethylene glycol ether of the diester of methylglucose and of stearic acid containing approximately 20 mol of ethylene oxide (INCI name: PEG-20 methyl glucose distearate), such as the product sold under the name Glucam E-20 distearate by the company Amerchol; the polyethylene glycol ether of the mixture of the monoester and diester of methylglucose and of stearic acid containing approximately 20 mol of ethylene oxide (INCI name: PEG-20 methyl glucose sesquistearate), and in particular the product sold under the name Glucamate SSE-20 by the company Amerchol and that sold under the name Grillocose PSE-20 by the company Goldschmidt, and mixtures thereof.

As sucrose esters, mention may, for example, be made of sucrose palmitostearate, sucrose stearate and sucrose monolaurate.

As fatty alcohol ethers, mention may, for example, be made of polyethylene glycol ethers of fatty alcohols containing from 8 to 30 carbon atoms, and in particular from 10 to 22 carbon atoms, such as the polyethylene glycol ethers of cetyl alcohol, of stearyl alcohol or of cetearyl alcohol (mixture of cetyl alcohol and stearyl alcohol). Mention may, for example, be made of ethers comprising from 1 to 200, and preferably from 2 to 100 oxyethylene groups, such as those having the INCI name Ceteareth-20 and Ceteareth-30, and mixtures thereof.

As sugar ethers, mention may in particular be made of alkylpolyglucosides, and for example decylglucoside, for instance the product sold under the name Mydol 10 by the company Kao Chemicals, the product sold under the name Plantaren 2000 by the company Henkel, and the product sold under the name Oramix NS10 by the company Seppic; caprylyl/capryl glucoside, for instance the product sold under the name Oramix CG 110 by the company Seppic or under the name Lutensol GD 70 by the company BASF; laurylglucoside, for instance the products sold under the names Plantaren 1200 N and Plantacare 1200 by the company Henkel; cocoglucoside, for instance the product sold under the name Plantacare 818/UP by the company Henkel; cetostearyl glucoside optionally as a mixture with cetostearyl alcohol, sold, for example, under the name Montanov 68 by the company Seppic, under the name Tego-Care CG90 by the company Goldschmidt and under the name Emulgade KE3302 by the company Henkel; arachidyl glucoside, for example in the form of the mixture of arachidyl alcohol and behenyl alcohol and of arachidyl glucoside, sold under the name Montanov 202 by the company Seppic; cocoylethylglucoside, for example in the form of the mixture (35/65) with cetyl alcohol and stearyl alcohol, sold under the name Montanov 82 by the company Seppic; and mixtures thereof.

The second composition B according to the invention can also contain, as emulsifier, an advantageous amount of amphiphilic polymers.

The term “amphiphilic polymer” is intended to mean all polymers comprising both a hydrophilic part and a hydrophobic part and having the property of forming a film separating two liquids of different polarity and thus making it possible to stabilize liquid-liquid dispersions of direct, inverse or multiple type. The amphiphilic polymers that are more particularly suitable reduce the water/oil surface tension to 10 mN/m, irrespective of the oil. These polymers are ionic (anionic or cationic) or amphoteric. They may be water-soluble or water-dispersible. The term “water-soluble” is intended to mean the fact that they can disperse in water in the form of a molecular solution. The term “water-dispersible” is intended to mean the fact that they can disperse in water in particulate form.

The amphiphilic polymers in accordance with the invention generally may have a number-average molecular weight ranging from 1000 to 20 000 000 g/mol, for example ranging from 20 000 to 8 000 000, and or for example from 100 000 to 700 000 g/mol. The amounts of amphiphilic polymers that may be used according to the invention may be selected from the group 0.01% to 20%, for example from 0.1% to 10%, or for example from 0.2% to 5% by weight, relative to the total weight of the composition containing said amphiphilic polymer.

Use may, for example be made of acrylate/C₁₀-C₃₀ alkyl acrylate copolymers such as the products sold under the names Pemulen TR1, Pemulen TR2 and Carbopol 1382 by the company Goodrich, or else mixtures thereof. Use may also be made of the acrylate/steareth-20 itaconate copolymers and acrylate/ceteth-20 itaconate copolymers sold under the names Structure 2001 and Structure 3001 by the company National Starch. By way of terpolymers that may be used, mention may be made of the methacrylic acid/methyl acrylate/behenyl dimethyl m-isopropenylbenzylisocyanate terpolymer ethoxylated with 40 EO (i.e. comprising 40 oxyethylene groups), sold by the company Amerchol under the name Viscophobe DB 1000 NP3-NP4.

Mention may also be made of crosslinked terpolymers of methacrylic acid, of ethyl acrylate and of polyethylene glycol (10 EO) stearyl ether (Steareth 10), in particular those sold by the company Allied Colloids under the name Salcare SC 80.

The anionic polymers that can be used according to the invention may be, for example, isophthalic acid or sulfoisophthalic acid polymers, and, for example, the phthalate/sulfoisophthalate/glycol copolymers (for example, diethylene glycol/phthalate/isophthalate/1,4-cyclohexanedimethanol) sold under the names Eastman AQ polymer (AQ35S, AQ38S, AQ55S, AQ48 Ultra) by the company Eastman Chemical.

Mention may also be made of amphiphilic polymers comprising at least one 2-acrylamidomethylpropanesulfonic acid (AMPS) unit.

The amphiphilic AMPS polymers according to the invention may, for example, be selected from the group consisting of in particular chosen from amphiphilic polymers of at least one acrylamidomethylpropanesulfonic acid (AMPS) monomer and of at least one ethylenically unsaturated comonomer comprising at least one hydrophobic part having from 7 to 30 carbon atoms, for example from 7 to 22 carbon atoms, or even from 12 to 22 carbon atoms.

The amphiphilic AMPS polymers according to the invention generally have a weight-average molecular weight ranging from 50 000 to 10 000 000 g/mol, for example from 100 000 to 8 000 000 g/mol, or for example from 100 000 to 7 000 000 g/mol.

They may be crosslinked or noncrosslinked.

By way of indication, and without this being limiting, mention may in particular be made of the copolymer of AMPS and of ethoxylated C₁₂-C₁₄ alcohol methacrylate (noncrosslinked copolymer obtained from Genapol LA-070 and from AMPS) (INCI name: ammonium acryloyldimethyltaurate/laureth-7 methacrylate copolymer) sold under the name Aristoflex LNC by the company Clariant, the copolymer of AMPS and of ethoxylated (25 EO) stearyl methacrylate (copolymer crosslinked with trimethylolpropane triacrylate, obtained from Genapol T-250 and from AMPS) (INCI name: ammonium acryloyldimethyltaurate/steareth-25 methacrylate crosspolymer) sold under the name Aristoflex HMS by the company Clariant, Aristoflex SNC (80/20 copolymer of AMPS/ethoxylated (8 mol EO) C₁₆/C₁₋₈ alcohol methacrylate; INCI name: ammonium acryloyldimethyltaurate/steareth-8/methacrylate copolymer) and Aristoflex HMB (copolymer of AMPS/ethoxylated (25 EO) behenyl methacrylate, crosslinked with trimethylolpropane triacrylate (TMPTA)).

For the W/O emulsions, emulsifiers that may be mentioned include, for example, dimethicone copolyols, such as the mixture of cyclomethicone and of dimethicone copolyol sold under the name DC 5225 C by the company Dow Corning, and alkyl dimethicone copolyols such as the lauryl methicone copolyol sold under the name Dow Corning 5200 Formulation Aid by the company Dow Corning and the cetyl dimethicone copolyol sold under the name Abil EM 90R by the company Goldschmidt, or the mixture of polyglyceryl-4 isostearate/cetyl dimethicone copolyol/hexyllaurate sold under the name Abil WE 09 by the company Goldschmidt. One or more coemulsifiers may also be added thereto. Advantageously, the coemulsifier can be chosen from the group comprising alkylated polyol esters. As alkylated polyol esters, mention may in particular be made of esters of glycerol and/or of sorbitan, and for example polyglyceryl isostearate, such as the product sold under the name Isolan GI 34 and Isolan GPS by the company Goldschmidt, sorbitan isostearate, such as the product sold under the name Arlacel 987 by the company ICI, and glyceryl sorbitan isostearate, such as the product sold under the name Arlacel 986 by the company ICI, and mixtures thereof.

As surfactant of W/O emulsions, use may also be made of a crosslinked elastomeric solid organopolysiloxane comprising at least one oxyalkylenated group, such as those obtained according to the protocol of Examples 3, 4 and 8 of document U.S. Pat. No. 5,412,004 incorporated herein by reference and of the examples of document U.S. Pat. No. 5,811,487 incorporated herein by reference, in particular the product of Example 3 (synthesis example) of patent U.S. Pat. No. 5,412,004 incorporated herein by reference, and such as those sold under the references KSG-210, KSG-310, KSG-320, KSG-330 and KSG-340 by the company Shin Etsu. Use may also be made of polyglycerolated silicone elastomers, in particular described in patent application US 2006/034875 incorporated herein by reference, for instance those sold under the names KSG-710, KSG-810, KSG-820, KSG-830, KSG-840 by the company Shin Etsu.

Polyisobutylene surfactants with an esterified succinic end group, such as those sold under the names Lubrizol 5603® and Chemcinnate 2000® by the companies Lubrizol and Chemron, are in particular suitable as emulsifiers that are suitable for obtaining a W/O emulsion.

According to an advantageous embodiment, the emulsion may be prepared by the phase inversion temperature technique (PIT emulsions). This technique makes it possible in particular to obtain an average size of the globules constituting the oily phase ranging from 0.1 to 4 μm (100 to 4000 nm).

Phase inversion emulsification is explained in detail in the work T. Förster, W von Rybinski, A. Wadle, Influence of microemulsion phases on the preparation of fine disperse emulsions, Advances in Colloid and interface sciences, 58, 119-149, 1995 mentioned herein for reference.

In the case of a PIT emulsion, the emulsifying system used in the second composition B according to the invention comprises one or more emulsifiers, the solubility of which in the oil increases as the temperature increases, and which has an HLB (hydrophilic lipophilic balance) ranging from 8 to 18, and preferably from 10 to 16. These emulsifiers are chosen from ethoxylated fatty alcohols, ethoxylated fatty acids, partial glycerides of ethoxylated fatty acids, polyglycerolated fatty acid triglycerides and ethoxylated derivatives thereof, and mixtures thereof.

The emulsifiers may be, for example, selected from the group consisting of ethoxylated fatty alcohols and ethoxylated fatty acids.

As ethoxylated fatty alcohols, mention may, for example, be made of the products of addition of ethylene oxide with lauryl alcohol, in particular those comprising from 9 to 50 oxyethylene groups (laureth-9 to laureth-50 in terms of INCI names); the products of addition of ethylene oxide with behenyl alcohol, in particular those comprising from 9 to 50 oxyethylene groups (beheneth-9 to beheneth-50 in terms of INCI names); the products of addition of ethylene oxide with cetearyl alcohol (mixture of cetyl alcohol and of stearyl alcohol), in particular those comprising from 9 to 30 oxyethylene groups (ceteareth-9 to ceteareth-30 in terms of INCI names); the products of addition of ethylene oxide with cetyl alcohol, in particular those comprising from 9 to 30 oxyethylene groups (ceteth-9 to ceteth-30 in terms of INCI names); the products of addition of ethylene oxide with stearyl alcohol, in particular those comprising from 9 to 30 oxyethylene groups (steareth-9 to steareth-30 in terms of INCI names); the products of addition of ethylene oxide with isostearyl alcohol, in particular those comprising from 9 to 50 oxyethylene groups (isosteareth-9 to isosteareth-50 in terms of INCI names); and mixtures thereof.

As ethoxylated fatty acids, mention may, for example, be made of the products of addition of ethylene oxide with lauric acid, palmitic acid, stearic acid or behenic acid, and mixtures thereof, in particular those comprising from 9 to 50 oxyethylene groups, such as the laurates of PEG-9 to PEG-50 (in terms of INCI names: PEG-9 laurate to PEG-50 laurate); the palmitates of PEG-9 to PEG-50 (in terms of INCI names: PEG-9 palmitate to PEG-50 palmitate); the stearates of PEG-9 to PEG-50 (in terms of INCI names: PEG-9 stearate to PEG-50 stearate); the palmitostearates of PEG-9 to PEG-50; the behenates of PEG-9 to PEG-50 (in terms of INCI names: PEG-9 behenate to PEG-50 behenate); and mixtures thereof.

Use may also be made of mixtures of these oxyethylenated derivatives of fatty alcohols and of fatty acids.

According to one embodiment, the emulsifying system of a second composition of the invention, which is in the form of an emulsion obtained according to the PIT technique, may contain as emulsifier at least one ethoxylated fatty alcohol, for example ceteth, ceteareth, beheneth, and mixtures thereof, and more particularly beheneth-10.

This emulsifying system may also contain one or more coemulsifiers. As coemulsifiers, mention may, for example, be made of fatty alcohols containing from 8 to 30 carbon atoms, for instance cetyl alcohol, stearyl alcohol or behenyl alcohol; fatty acids containing from 8 to 30 carbon atoms, for instance palmitic acid, stearic acid or behenic acid; fatty esters of glycerol, for instance glyceryl stearate; oxyethylenated derivatives of these fatty alcohols, fatty acids and fatty esters of glycerol, comprising 2 to 8 ethylene oxide groups, and mixtures thereof.

As examples of oils that may be used in a composition B according to the invention, mention may be made of:

hydrocarbon-based oils of animal origin, such as perhydrosqualene;

hydrocarbon-based oils of plant origin, such as liquid triglycerides of fatty acids containing from 4 to 10 carbon atoms, for instance triglycerides of heptanoic acid or of octanoic acid, or else, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides, such as those sold by the company Stearineries Dubois or those sold under the names Miglyol 810, 812 and 818 by the company Dynamit Nobel, jojoba oil, shea butter oil;

synthetic esters and ethers, for example, of fatty acids, such as the oils of formulae R₁COOR₂ and R₁OR₂ in which R₁ represents the residue of a fatty acid containing from 8 to 29 carbon atoms, and R₂ represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, for instance propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or nonvolatile liquid paraffins, and derivatives thereof, isohexadecane, isododecane, petroleum jelly, polydecenes, hydrogenated polyisobutene such as Parléam® oil;

natural or synthetic essential oils such as, for example, eucalyptus oil, lavandin oil, lavender oil, vetiver oil, Litsea cubeba oil, lemon oil, sandalwood oil, rosemary oil, chamomile oil, savory oil, nutmeg oil, cinnamon oil, hyssop oil, caraway oil, orange oil, geraniol oil, cade oil and bergamot oil;

fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluorooils such as those described in document JP-A-2-295912 incorporated herein by reference;

silicone oils such as volatile or nonvolatile polydimethylsiloxanes (PDMSs) containing a linear or cyclic silicone chain, which are liquid or pasty at ambient temperature, for example, cyclopolydimethylsiloxanes (cyclomethicones) such as cyclohexasiloxane and cyclopentasiloxane; polydimethylsiloxanes comprising alkyl, alkoxy or phenyl groups, which are pendent or at the end of a silicone chain, these groups containing from 2 to 24 carbon atoms; phenylsilicones such as phenyl trimethicones, phenyl dimethicones, phenyl trimethylsiloxydiphenylsiloxanes, diphenyl dimethicones, diphenylmethyldiphenyltrisiloxanes, 2-phenylethyltrimethylsiloxysilicates and polymethylphenylsiloxanes;

mixtures thereof.

The term “hydrocarbon-based oil” in the list of oils mentioned above is intended to mean any oil predominantly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes such as lanolin, beeswax, carnauba wax or candelilla wax, paraffin wax, lignite wax or microcrystalline waxes, ceresine or ozokerite, synthetic waxes such as polyethylene waxes, Fischer-Tropsch waxes; gums such as silicone gums (dimethiconol).

The compositions B according to the invention may comprise a volatile oil.

For the purpose of the invention, the term “volatile oil” is intended to mean an oil that is capable of evaporating on contact with keratin materials in less than one hour, at ambient temperature and atmospheric pressure. The volatile organic solvent(s) and the volatile oils of the invention are volatile organic solvents and volatile cosmetic oils that are liquid at ambient temperature, with a nonzero vapor pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

As volatile oils, mention may be made, inter alia, of cyclic or linear silicones containing from 2 to 6 silicon atoms, such as cyclohexasiloxane, dodecamethylpentasiloxane, decamethyltetrasiloxane, butyltrisiloxane and ethyltrisiloxane. It is also possible to use branched hydrocarbons, for instance isododecane and also volatile perfluoroalkanes such as dodecafluoropentane and tetradecafluorohexane, sold under the names PF 5050® and PF 5060® by the company 3M, and perfluoromorpholine derivatives, such as 4-trifluoromethylperfluoromorpholine sold under the name PF 5052® by the company 3M.

The amount of oily phase present in the compositions B according to the invention may range, for example, from 0.01% to 50% by weight, for example 0.1% to 30% by weight, relative to the total weight of the composition.

The compositions B according to the invention may also comprise at least one dyestuff selected, for example, from the group consisting of pigments, pearlescent agents, dyes and materials with an effect, and mixtures thereof.

These dyestuffs may be present in a content ranging from 0.01% to 50% by weight, relative to the total weight of the composition, for example from 0.01% to 30% by weight.

The compositions B according to the invention may also comprise an organic or inorganic filler, for example in a content ranging from 0.01% to 50% by weight, relative to the total weight of the composition, for example ranging from 0.01% to 30% by weight. These fillers may be or organic and of any shape, platelet-shaped, spherical or oblong, irrespective of the crystallographic form (for example, lamellar, cubic, hexagonal, orthorhombic or amorphous). Mention may be made of silica, talc, mica, kaolin, lauroyllysine, starch, boron nitride, PTFE powders, PMMA powders, methylsilsesquioxane resin powders (such as Tospearl 145A from GE Silicone), hollow hemispherical silicone resin particles (for instance NLK 500, NLK 506 and NLK 510 from Takemoto Oil and Fat), barium sulfate, precipitated calcium carbonate, magnesium carbonate, magnesium hydrogen carbonate, hydroxyapatite, glass or ceramic microcapsules, and metal soaps derived from organic carboxylic acids containing from 8 to 22 carbon atoms, for example from 12 to 18 carbon atoms, for example zinc stearate, magnesium stearate or lithium stearate, zinc laurate or magnesium myristate.

The compositions B according to the invention may also contain various adjuvants commonly used in the cosmetics field, such as sequestering agents; UV screening agents; fragrances; and thickeners and gelling agents.

Among the UV screening agents, mention may be made of organic and/or inorganic screening agents which are active in the UVA and/or UVB range, and which are hydrophilic and/or lipophilic and/or insoluble in the cosmetic solvents commonly used.

According to the fluidity of composition B that it is desired to obtain, one or more gelling agents, in particular hydrophilic, i.e. water-soluble or water-dispersible, can be incorporated into the composition.

As hydrophilic gelling agents, mention may, for example, be made of water-soluble or water-dispersible thickening polymers. The latter may, for example, be selected from the group consisting of: modified or unmodified carboxyvinyl polymers, such as the products sold under the names Carbopol (INCI name: carbomer) and Pemulen (INCI name: acrylates/C10-30 alkyl acrylate crosspolymer) by the company Goodrich; polyacrylates and polymethacrylates, such as the products sold under the names Lubrajel and Norgel by the company Guardian or under the name Hispagel by the company Hispano Chimica; polyacrylamides; optionally crosslinked and/or neutralized 2-acrylamido-2-methylpropanesulfonic acid polymers and copolymers, such as the poly(2-acrylamido-2-methylpropanesulfonic acid) sold by the company Clariant under the name Hostacerin AMPS (INCI name: ammonium polyacryldimethyltauramide); crosslinked anionic copolymers of acrylamide and of AMPS which are in the form of a W/O emulsion, such as those sold under the name Sepigel 305 (INCI name: polyacrylamide/C13-14 isoparaffin/laureth-7) and under the name Simulgel 600 (INCI name: acrylamide/sodium acryloyldimethyltaurate copolymer/isohexadecane/polysorbate 80) by the company Seppic; polysaccharide biopolymers such as xanthan gum, guar gum, locust bean gum, gum acacia, scleroglucans, chitin derivatives and chitosan derivatives, carragheenans, gellans, alginates, celluloses such as microcrystalline cellulose, carboxymethylcellulose, hydroxymethylcellullose and hydroxypropylcellulose, and mixtures thereof. Use may also be made of maleic anhydride copolymers such as the methyl vinyl ether/maleic anhydride copolymer crosslinked with 1,9-decadiene (INCI name: PVM/MA decadiene crosspolymer) sold under the name Stabileze OM® or 06® by the company ISP. The amphiphilic polymers mentioned above may also be used as hydrophilic gelling agents.

As lipophilic gelling agents, mention may, for example, be made of modified clays such as modified magnesium silicate (bentone gel VS38 from Rheox), or the hectorite modified with distearyl dimethyl ammonium chloride (INCI name: disteardimonium hectorite) sold under the name Bentone 38 CE by the company Rheox.

A composition B according to the invention may also comprise one or more additional active cosmetic or therapeutic agent(s).

The term “active agent” is intended to mean any compound having a beneficial effect on the keratin material to which the final product is applied, in particular on the skin.

By way of examples, and without this list being limiting, active agents having a cosmetic or dermatological application that may be mentioned include:

anti-UV agents, anti-aging/antiwrinkle agents (such as antiglycation agents, agents for stimulating the synthesis of dermal or epidermal macromolecules and/or preventing their degradation, agents for stimulating fibroblast and/or keratinocyte proliferation or for stimulating keratinocyte differentiation, muscle relaxants), moisturizers, desquamating agents, antipollution agents and free-radical scavengers, antiperspirants and deodorants, tensioning agents, slimming agents, agents that act on the microcirculation, agents that act on the energy metabolism of cells, tensioning agents, depigmenting or propigmenting agents, desquamating agents, antiseborrheic agents, anti-acne agents or anti-inflammatories/anti-irritants.

Mention may also be made of all active agents known for their activity on skin aging, for instance c-glycosides and derivatives such as C-β-D-xylopyranoside-2-hydroxypropane or C-α-D-xylopyranoside-2-hydroxypropane, and in particular C-β-D-xylopyranoside-2-hydroxypropane in the form of a solution at 30% with respect to active material in a water/propylene glycol mixture (60/40% by weight), such as the product manufactured by Chimex under the trade name Mexoryl SBB®, keratolytic or prodesquamating agents, for example α-hydroxy acids, β-hydroxy acids, α-keto acids, retinoids and their esters, retinol, retinoic acid and its derivatives; vitamins C, B3 or PP, B5, E and the derivatives of these vitamins, and in particular their esters, vitamin K and its derivatives (K1, K2, etc), adenosine and its derivatives; free-radical scavengers; DHEA and its derivatives; coenzyme Q10; whitening and depigmenting agents such as kojic acid, para-aminophenol derivatives, arbutin and their derivatives, and mixtures thereof.

The active agents may also be active hair treatment agents such as, for example, agents for inhibiting hair loss, agents for stimulating hair growth and antidandruff agents.

As indicated above, the second composition B of the invention may be in any of the gallenical forms that may be envisioned, provided that it is suitable for the dissolution of the film-type composition.

According to one embodiment-, a composition B according to the invention may have the form of an aqueous or aqueous-alcoholic solution; of a dispersion of the lotion or serum type; of a water-in-oil, oil-in-water or multiple emulsion; or of a suspension.

A second composition B according to the invention may advantageously be in the form of a cleansing, protecting, treating or care composition for the face, for the hands, for the feet, for the major anatomical folds or for the body (for example, day creams, night cream, makeup-removing cream, antisun composition, protective body milks or bodycare milks, aftersun milks, skincare lotion, gel or mousse, cleansing lotions); a composition for making up the body or the face, such as a foundation; a bath composition; a deodorant composition; an aftershave composition.

A composition B according to the invention may also be in the form of a haircare composition, in particular a shampoo, a treating lotion, a hairstyling cream or gel, hair restructuring lotions, an anti-hairloss lotion or gel, or an antiparasitic shampoo.

The mixture of the two respective compositions A and B according to the invention may be applied by any means which allows even spreading, and for example by using cotton wool, a cotton bud, a brush, a gauze, a spatula or a pad, and may be removed by rinsing with water or using a mild detergent.

The examples shown hereinafter are presented by way of nonlimiting illustration of the field of the invention.

### EXAMPLE 1

### Preparation of Compositions a in the Form of a Film, in Accordance with the Invention

The composition of the solution used for the formation of a first composition of films referred to as Film 1 is the following:

| | Film 1 |
|---|---|
| Name | Amount as % |
| Polyvinyl alcohol (Celvol 540 PVA) | 2.0 |
| Glucose | 1.0 |
| Hydroxypropylcellulose (Klucel EF) | 3.1 |
| Water | 81.3 |
| Carragheenan | 1.0 |
| Bifidobacterium longum | 3 |
| Esterified corn starch (Dry Flo Plus) | 1.6 |
| Glycerol | 3.1 |
| Polyethylene glycol 400 | 1.9 |
| PEG-12 Dimethicone (Silsoft 880) | 2.0 |

The film is prepared in two steps. In a first step, the mixture is prepared and, in a second step, layering of this mixture is carried out, coupled with drying thereof so as to form the film.

More specifically, the two steps are as follows:

1^{st} step: the polymer(s) is (are) dispersed in water and the whole mixture is left to stir at a speed and for a period of time that are necessary to obtain a homogeneous gel. This step lasts 3 hours with vigorous stirring (1200 to 1600 rpm) using a turbine mixer (Rayneri).

2^{nd} step: the oxyethylenated PDMS, the probiotics and the carragheenan are dispersed. Once the mixture is homogenized, the starch, the PEG 400 and the glycerol are added, stirring being carried out at the same speed as above. This step lasts 20 minutes.

The mixture is layered onto the nonsiliconized face of a sheet of Silphan S100 M44A, using a film drawer. The apparatus used is an automatic film-applying apparatus from Braive Instruments. The wet thickness chosen is in general 500 μm. After drying in a tunnel with circulation of hot air at a temperature of approximately 50° C., the film obtained is cut into the desired shape, for example into the form of 25 mm-sided square blocks.

After drying, an anhydrous film approximately 50 μm thick is obtained, the composition of which is:

| | Film 1 |
|---|---|
| Name | Amount as % |
| Polyvinyl alcohol (Celvol 540 PVA) | 10.7 |
| Glucose | 5.34 |
| Hydroxypropylcellulose (Klucel EF) | 16.57 |
| Carragheenan | 5.35 |
| Bifidobacterium longum | 16 |
| Esterified corn starch (Dry Flo Plus) | 8.55 |
| Glycerol | 16.57 |
| Polyethylene glycol 400 | 10.2 |
| PEG-12 Dimethicone (Silsoft 880) | 10.7 |

Three other film-type compositions as defined hereinafter are also obtained on the basis of the above protocol using, as microorganisms, the microorganisms specified hereinafter for each of them.

| | Film 2 | Film 3 | Film 4 | Film 5 |
|---|---|---|---|---|
| | Amount | Amount | Amount | Amount |
| Name | as % | as % | as % | as % |
| Polyvinyl alcohol | 10.7 | 10.7 | 10.7 | 10.7 |
| (Celvol 540 PVA) | | | | |
| Glucose | 5.34 | 5.34 | 5.34 | 5.34 |
| Hydroxypropylcellulose | 16.57 | 16.57 | 16.57 | 16.57 |
| (Klucel EF) | | | | |
| Carragheenan | 5.35 | 5.35 | 5.35 | 5.35 |
| Lactobacillus paracasei | 16 | | | |
| Lactobacillus johnsonii | | 16 | | |
| Bifidobacterium lactis | | | 16 | |
| Saccharomyces cerevisae | | | | 16 |
| Esterified corn starch | 8.55 | 8.55 | 8.55 | 8.55 |
| (Dry Flo Plus) | | | | |
| Glycerol | 16.57 | 16.57 | 16.57 | 16.57 |
| Polyethylene glycol 400 | 10.2 | 10.2 | 10.2 | 10.2 |
| PEG-12 Dimethicone | 10.7 | 10.7 | 10.7 | 10.7 |
| (Silsoft 880) | | | | |

### EXAMPLE 2

### Product in accordance with the invention

First Composition in Film Form

Film No. 1 of example 1 is used in the form of a small 25 mm-sided square.

Second composition of moisturizing serum type
Phase A

| | |
|---|---|
| PVM/MA decadiene crosspolymer | 0.20 g |
| (Stabileze from the company ISP) | |
| Xanthan gum | 0.20 g |
| Methylparaben | 0.20 g |
| Phenoxyethanol | 0.35 g |
| Water | qs 100 |

Phase B

| | |
|---|---|
| Triethanolamine | 0.20 g |
| Polyacrylamide and C13-C14 isoparaffin and laureth-7 | 1.00 g |
| (Sepigel 305 from the company Seppic) | |
| Diazolidinyl urea | 0.30 g |
| Glycerol | 7.00 g |

Protocol for preparing the serum: phase A is heated to approximately 75° C., with stirring, and then phase B, prepared beforehand, is poured into phase A. The heating is then stopped while at the same time maintaining the stirring until the mixture returns to ambient temperature. Gentle stirring is then maintained for 30 minutes.

The aqueous composition (serum) and one or more films of example 1 are provided in the form of a kit.

At the time of use, the consumer mixes one or more films of example 1 with a dose of between 100 and 500 mg of the aqueous composition, for about ten seconds, in the hollow of his or her hand with the fingers.

The consumer lightly massages the surface to be treated so as to promote spreading of the product thus obtained, over the skin.

The final product obtained can be applied to the face or the body. The skin is then moisturized.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A composition which is in the form of a water-soluble anhydrous film comprising (i) at least one water-soluble or water-dispersible film-forming polymer, and (ii) at least one live, in particular probiotic, microorganism, and comprising at least one oxyalkylenated polydimethylsiloxane derivative.

2. The composition as claimed in claim 1, the film of which has a thickness of from 10 μm to 1000 μm.

3. The composition as claimed in claim 1 containing less than 15% by weight of water.

4. The composition as claimed in claim 1, in which the film-forming polymer has a solubility in water, measured at 25° C., at least equal to 0.1 g/liter.

5. The composition as claimed in claim 1, in which the film-forming polymer is selected from the group consisting of vinyl polymers, cellulosic derivatives, starches and their derivatives, optionally modified polymers of natural origin, polymers derived from chitin or from chitosan, protein polymers, acrylic phosphorylcholin copolymers and anion-cation complexes, and mixtures thereof.

6. The composition as claimed in claim 1, in which the film-forming polymer is selected from the group consisting of vinyl polymers and cellulosic derivatives, and mixtures thereof.

7. The composition as claimed in claim 6, in which the film-forming polymer is selected from the group consisting of polyvinyl acetate, hydroxypropylcellulose and hydroxypropylmethylcellulose, and mixtures thereof.

8. The composition as claimed in claim 1, in which the amount of film-forming polymer(s) ranges from 10% to 95% by weight relative to the total weight of the composition.

9. The composition as claimed in claim 1, in which said microorganism is selected from the group consisting of ascomycetes such as Saccharomyces, Yarrowia, Kluyveromyces, Torulaspora, Schizosaccharomyces pombe, Debaromyces, Candida, Pichia, Aspergillus and Penicillium, and bacteria of the genus Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus or Lactobacillus, and mixtures thereof.

10. The composition as claimed in claim 1, in which said microorganism is a probiotic microorganism.

11. The composition as claimed in claim 10, in which said probiotic microorganism is selected from the group consisting of lactic acid bacteria, bifidobacteria, yeasts and sporulated bacteria, and mixtures thereof.

12. The composition as claimed in claim 11, in which said probiotic microorganism comprises at least one microorganism of the Lactobacillus or Bifidobacterium group.

13. The composition as claimed in claim 12, in which the microorganism is selected from the group consisting of Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium lactis, Bifidobacterium infantis, Bifidobacterium adolescentis or Bifidobacterium pseudocatenulatum, and mixtures thereof.

14. The composition as claimed in claim 1, comprising from 10-1 cfu/g to 1015 cfu/g of live microorganism(s).

15. The composition as claimed in claim 1, comprising at least one probiotic microorganism combined with at least one live microorganism of skin flora and/or a nonphotosynthetic filamentous bacterium or one of its extracts.

16. The composition as claimed in claim 1, also comprising at least one polysaccharide thickener.

17. A kit for formulating a cosmetic product, comprising:
(i) at least a first composition referred to as A as defined in claim 1, and
(ii) a second composition referred to as B, comprising at least one physiologically acceptable medium.

18. The kit as claimed claim 17, in which the product is for oral administration.

19. The kit as claimed in claim 17, in which the product is for topical application.

20. The kit as claimed claim 17, in which the second composition contains at least one liquid fatty phase.

21. The kit as claimed in claim 17, in which the second composition is in the form of an emulsion.

22. The kit as claimed in claim 17, in which the second composition comprises at least one compound selected from the group consisting of thickeners, gelling agents, emulsifiers, dyestuffs and/or organic or inorganic fillers.

23. The kit as claimed in claim 19, in which the second composition comprises at least one active agent having a cosmetic or dermatological application.

24. A cosmetic product obtained by incorporating at least one composition as defined in claim 1 into a second composition comprising at least one physiologically acceptable medium.

25. A process for cosmetic care of keratin materials, comprising the topical or oral administration of a product as defined in claim 17.
